(19) United States
(12) Patent Application Publication
(10) Pub. No.: US 2021/0113743 A1
(43) Pub. Date: Apr. 22, 2021

(54) MULTILAYER MEDICAL TUBING WITH ENHANCED ADHESION

(71) Applicant: CAREFUSION 303, INC., San Diego, CA (US)

(72) Inventors: Zehra SEVINC, Vernon Hills, IL (US); James FREASIER, Franklin Lakes, NJ (US)

(21) Appl. No.: 16/656,330

(22) Filed: Oct. 17, 2019

(51) Int. Cl. A61L 29/08; A61L 29/04; A61L 29/14

(52) U.S. Cl. CPC A61L 29/085; A61M 5/14; A61L 29/14; A61L 29/041

(57) ABSTRACT

Co-extruded tubing for administration of intravenous fluids has at least one adhesion layer comprising a non-hydrogenated styrene-based thermoplastic elastomer directly adjacent at a balance layer comprising a hydrogenated styrene-based thermoplastic elastomer. Such tubing can be used to form infusion sets.

### TECHNICAL FIELD

The present disclosure generally relates to tubing and, in particular, to medical tubing for administration of medical fluid by infusion.

### BACKGROUND

The assembly of tubing, valves, fittings, and needles that connect a fluid container or an infusion pump to a patient may be referred to as an “IV set.” Polyvinyl chloride (PVC) is widely used for such tubing. However, a large amount of plasticizer is needed to make PVC tubing flexible, and such plasticizers can be a concern for certain medical applications due to potential extraction and leaching thereof into the fluid transferred through the PVC tubing. Hence, there is desire to use polyvinyl-chloride-free materials for medical tubing.

Hydrogenated products of styrene-based thermoplastic elastomers (s-TPE) and saturated polyolefins can have good flexibility, processability and can be physiologically inert and are finding applications as viable substitutes for PVC tubing. Such materials, however, are difficult to bond to other medical components, particularly components made of different polymeric materials, due to their relative chemical inertness. Hence, a more significant issue in manufacturing infusion sets using tubing made from hydrogenated sTPE is to consistently join such and infusion components to obtain a secure and/or leak free joint with desired fluid flow.

Accordingly, there is a continuing need to develop medical tubing that can be used with infusion sets without the disadvantages of PVC materials and with the advantages of s-TPE.

### SUMMARY

Aspects of the subject technology relate to tubing and, in particular, to medical tubing for administration of medical fluid by infusion in which one layer, an adhesion layer, and another layer, a balance layer. In accordance with certain aspects, a co-extruded tubing for administration of intravenous fluids can have at least one adhesion layer directly adjacent a balance layer. The adhesion layer can comprise a non-hydrogenated styrene-based thermoplastic elastomer and the other layer, a balance layer, can comprise a hydrogenated styrene-based thermoplastic elastomer. The adhesion layer can be either an inner layer or an outer layer of the tubing or both an inner and outer layer of the tubing depending on how the tubing is intended to be connected to with another component.

The subject technology also relates to an infusion set comprising the co-extruded tubing bound to one or more medical connectors and a method of preparing an infusion set comprising the co-extruded tubing.

Embodiments include one or more of the following features individually or combined. For example, the adhesion layer can comprise a styrene-diene block copolymer such as a styrene-butadiene copolymer. In other embodiments, the balance layer can comprise a styrene-ethylene copolymer, styrene-propylene copolymer, styrene-butylene copolymer, hydrogenated styrene-butadiene copolymer, hydrogenated styrene-isoprene copolymer or combinations thereof. The balance layer can also be a blend of a hydrogenated styrene based thermoplastic elastomer with a hydrogenated or saturated polyolefin. In certain embodiments, it is advantageous for the co-extruded tubing to be polyvinyl chloride free. In still further embodiments, the medical connector comprises an acrylic-based polymer. In some embodiments, an infusion set can be prepared by comprising the co-extruded tubing applying a thin layer of solvent on one or both of a surface of the tubing and a surface of a medical connector, and holding the surface of the tubing and the surface of the medical connector together until a seal is formed. The solvent can be applied to an inner diameter or outer diameter, or both of the tubing as the surface(s) of the tubing.

Additional advantages of the subject technology will become readily apparent to those skilled in this art from the following detailed description, wherein only certain aspects of the subject technology are shown and described, simply by way of illustration. As will be realized, the subject technology is capable of other and different configurations, and its several details are capable of modifications in various other respects, all without departing from the subject technology. Accordingly, the drawings and description are to be regarded as illustrative in nature, and not as restrictive.

### BRIEF DESCRIPTION OF THE DRAWINGS

The accompanying drawing, which is included to provide further understanding and is incorporated in and constitute a part of this specification, illustrate disclosed embodiments and together with the description serve to explain the principles of the disclosed embodiments. In the drawings:

FIG. 1 illustrates a cross-section of a two-layer tubing in accordance with an aspect of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Aspects of the subject technology relate to tubing and, in particular, to medical tubing for administration of medical fluid by infusion. In accordance with certain aspects, a co-extruded tubing for administration of intravenous fluids has at least one adhesion layer directly adjacent a balance layer. The adhesion layer can comprise non-hydrogenated, e.g. unsaturated, styrene based polymeric material whereas the balance layer can comprise a hydrogenated styrene polymeric material. Due to the chemical similarity of the adhesion and balance layers, the materials are relatively compatible and do not require a tie layer therebeween. In addition, the adhesion layer can be either an inner or an outer layer or both an inner and outer layer of the co-extruded tubing depending on how the tubing is to be connected with another component such as a connector in an IV set.

Exemplary adhesion layer materials include, without limitation, unsaturated styrene-based thermoplastic elastomers such as styrene-diene copolymers and block copolymers, styrene-butadiene copolymers such as styrene-butadiene-styrene block copolymers, styrene-butadiene-styrene-styrene block copolymers, styrene-isoprene copolymers such as styrene-isoprene-styrene block copolymers, etc.

Exemplary balance layer materials include, without limitation, hydrogenated styrene-based thermoplastic elastomers (s-TPE) such as styrene-ethylene, -propylene, and/or butylene block copolymers, hydrogenated styrene-butadiene or isoprene copolymers, styrene-ethylene-butylene-styrene thermoplastic elastomer (SEBS), styrene-butadiene copolymer (SBC), styrene-butadiene-styrene-styrene block copolymer (SBSS), styrene-isoprene-styrene (SIS), styrene-ethylene/propylene-styrene (SEPS) and combinations such as blends of the foregoing. Such exemplary balance materials can be optically transparent, heat stable, chemical and radiation sterilizable (e.g., sterilized with ethylene oxide or gamma radiation) and physiologically inert. Such exemplary balance materials are also environmentally compatible and can be incinerated with minimal contaminates. In addition, the balance layer can include a blend of the hydrogenated styrene-based thermoplastic elastomer with a hydrogenated or saturated polyolefin such as polypropylene, e.g., homo, block and/or random polypropylene such as random copolymer polypropylene (RCPP), etc.

Tubing of the present discloser can have an inner diameter for flow of fluid therethrough ranging from about 0.1 mm to about 3.5 mm, e.g., from about 0.5 mm to about 2.5 mm and an overall sidewall thickness ranging from 0.1 mm to 2 mm, such as from about 0.4 mm to about 1 mm. In some aspects of the present disclosure, the balance layer can comprise 95%, 70%, 50% and 5% of the side wall thickness and the adhesion layer can comprise 5%, 30%, 50%, and 95% of the sidewall thickness. In other aspects of the present disclosure, the adhesion layer has a thickness that is from about 5% to about 1900%, e.g., from about 40% to about 100%, of a thickness of the balance layer.

Tubing of the present disclosure is particularly useful with intravenous assemblies, gravity containers and/or infusion pumps for the transport of intravenous fluid to a patient. An assembly of tubing, valves, fittings, and needles that connect a fluid container to a patient intravenously may be referred to as an “IV set”. Infusion pumps are medical devices that may be used to administer intravenous (IV) fluids. Such assemblies, containers and pumps employ tubing bound to one or more medical connectors and tubing of the present disclosure is useful as such.

For example, tubing according to the present disclosure can be readily mated and bond to a medical connector or other medical component by applying a thin layer of solvent or adhesive on one or both of the contacting surfaces and then the two surfaces are brought together and held in place until a seal is formed. The contacting surface of the tubing can either be at the inner diameter, the outer diameter, or both diameters depending on whether the adhesion layer is an inner layer, outer layer or both. The solvent/adhesive is applied either internally, externally, or both. Medical connectors that can benefit from tubing of the present disclosure include, without limitation, connectors made of acrylic-based polymeric materials such as acrylonitrile-butadiene-styrene (ABS), methyl methacrylate-acrylonitrile-butadiene-styrene (mABS), acrylic-polycarbonate based materials, polyester, polycarbonate, acrylic, etc.

In an embodiment of the present application, tubing according to the present disclosure can be readily mated and bond to a medical connector by solvent boding. As described in the preceding paragraph, solvent boding includes applying a thin layer of solvent on one or both of the contacting surfaces, e.g. a surface of the tubing and a surface of the medical connector, and then the two surfaces are brought together and held in place until a seal is formed. The contacting surface of the tubing can either be at the inner diameter, the outer diameter, or both diameters depending on whether the adhesion layer is an inner layer, outer layer or both. The solvent is applied either internally, externally, or both.

Solvents that can be used in solvent bonding tubing of the present disclosure include, without limitation, one or more of an alcohol, such as methanol, ethanol, isopropanol, propanol, butanol, pentanol, benzyl alcohol, a glycol such as diethylene glycol, propylene glycol, an ether, such as dioxolane, tetrahydrofuran, 1,3-dioxane, a ketone, such as acetone, methyl ethyl ketone, cyclohexanone, an ester such as ethyl acetate, ethyl formate, an amide such as dimethyl formamide, dimethyl and diethyl acetamide, N-methyl pyrrolidone, a sulfoxide, such as dimethyl sulfoxide, a hydrocarbon such as hexanes, toluene, a chlorinated solvents such as methylene chloride, etc.

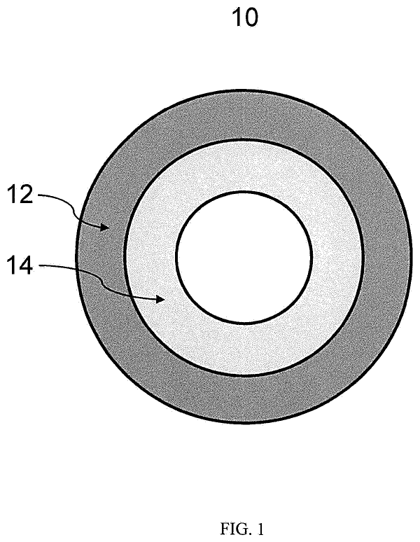

FIG. 1 illustrates a cross-section of a co-extruded double layer tubing configuration in accordance with an aspect of the present disclosure. As shown in the FIGURE, tubing, e.g., medical tubing 10, can include an adhesive outer layer (12) and a core or balance inner layer (14). The layers can be co-extruded such that outer layer 12 directly contacts inner layer 14 along the tubing length with no tie layer therebetween. In accordance with the present disclosure, adhesive outer layer 12 can be made of styrene-butadiene copolymer and balance inner layer 14 can be made of a styrene-ethylene, -propylene, and/or butylene block copolymer or blend thereof. For this example, having the balance layer inside of tubing provides advantages of better drug/chemical compatibility, less extractable and leachable components to fluid transported through the tubing.

The adhesion layer as an outer layer of the tubing (10) allows solvent bonding of the tubing to pocket joints of connectors such as connectors comprising rigid acrylic-based materials.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A co-extruded tubing for administration of intravenous fluids has at least one adhesion layer comprising a non-hydrogenated styrene-based thermoplastic elastomer directly adjacent a balance layer comprising a hydrogenated styrene-based thermoplastic elastomer.

2. The co-extruded tubing of claim 1, wherein the at least adhesion layer comprises a styrene-diene block copolymer.

3. The co-extruded tubing of claim 1, wherein the at least one adhesion layer comprises a styrene-butadiene copolymer.

4. The co-extruded tubing of claim 1, wherein the balance layer comprise a styrene-ethylene copolymer, styrene-propylene copolymer, styrene-butylene copolymer, hydrogenated styrene-butadiene copolymer, hydrogenated styrene-isoprene copolymer or combinations thereof.

5. The co-extruded tubing of claim 1, wherein the balance layer further comprises a hydrogenated or saturated polyolefin.

6. The co-extruded tubing of claim 1, wherein the at least one adhesion layer is an outer layer of the tubing and the balance layer is an inner layer of the tubing.

7. The co-extruded tubing of claim 1, wherein the balance layer is an outer layer of the tubing and the at least one adhesion layer is an inner layer of the tubing.

8. The co-extruded tubing of claim 1, wherein the adhesion layer has a thickness that is from about 5% to about 1900% of a thickness of the balance layer.

9. The co-extruded tubing of claim 1, wherein the tubing is polyvinyl chloride free.

10. An infusion set comprising the co-extruded tubing of claim 1 bound to a medical connector.

11. The infusion set of claim 10, wherein the medical connector comprises an acrylic-based polymer.

12. The infusion set of claim 10, wherein the at least adhesion layer comprises a styrene-diene block copolymer.

13. The infusion set of claim 10, wherein the balance layer comprise a styrene-ethylene copolymer, styrene-propylene copolymer, styrene-butylene copolymer, hydrogenated styrene-butadiene copolymer, hydrogenated styrene-isoprene copolymer or combinations thereof.

14. The infusion set of claim 10, wherein the at least one adhesion layer is an outer layer of the tubing and the balance layer is an inner layer of the tubing.

15. A method of preparing an infusion set comprising the co-extruded tubing of claim 1, the method comprising:
applying a thin layer of solvent on one or both of a surface of the tubing and a surface of a medical connector, and
holding the surface of the tubing and the surface of the medical connector together until a seal is formed.

16. The method of claim 15, wherein the solvent is applied to an inner diameter of the tubing as the surface of the tubing.

17. The method of claim 15, wherein the solvent is applied to an outer diameter of the tubing as the surface of the tubing.

18. The method of claim 15, wherein the medical connector comprises an acrylic-based polymer.

19. The method of claim 15, wherein the at least adhesion layer comprises a styrene-diene block copolymer.

20. The method of claim 15, wherein the at least adhesion layer comprises a styrene-diene block copolymer, the balance layer comprise a styrene-ethylene copolymer, styrene-propylene copolymer, styrene-butylene copolymer, hydrogenated styrene-butadiene copolymer, hydrogenated styrene-isoprene copolymer or combinations thereof, and the medical connector comprises an acrylic-based polymer.
